(19) United States
(12) Patent Application Publication
(10) Pub. No.: US 2003/0180304 A1
(43) Pub. Date: Sep. 25, 2003

(54) Secretory tyrosine phosphatases from mycobacteria

(76) Inventors: Axel Ullrich, Munchen (DE); Anil Koul, Munich (DE)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/257,935

(22) PCT Filed: Apr. 19, 2001
(86) PCT No.: PCT/EP01/04463

(30) Foreign Application Priority Data
Apr. 20, 2000 (EP) 00108682.6

(51) Int. Cl. A61K 39/40

(52) U.S. Cl. 424/168.1

(57) ABSTRACT

The present invention relates to a composition capable of inhibiting or preventing mycobacterial growth. In one embodiment of the present invention the composition comprises an inhibitor of secretory tyrosine phosphatases from mycobacteria as an active agent. In a further embodiment the composition comprises a secretory tyrosine phosphatase from mycobacteria or an immunogenic fragment thereof or a nucleic acid encoding a secretory tyrosine phosphatase from mycobacteria or an immunogenic fragment thereof.

### Description

The present invention relates to a composition capable of inhibiting or preventing mycobacterial growth. In one embodiment of the present invention the composition comprises an inhibitor of secretory tyrosine phosphatases from mycobacteria as an active agent. In a further embodiment the composition comprises a secretory tyrosine phosphatase from mycobacteria or an immunogenic fragment thereof or a nucleic acid encoding a secretory tyrosine phosphatase from mycobacteria or an immunogenic fragment thereof.

With one third of the world population infected with tubercle bacilli and three million deaths every year, tuberculosis (TB) continues to be the most important cause of death (Snider et al., 1994). TB is spreading rapidly throughout the world with the advent of AIDS and development of resistance against most of the antibiotics used in the treatment of this disease. The need to focus on the goal of global tuberculosis control through basic and applied research in its diagnosis, treatment and prevention cannot be overemphasized. There is an urgent need for developing rapid and inexpensive means of diagnosis, understanding the nature of protective immunity and developing new drugs and vaccines. An important prerequisite for rapid development in these areas is the understanding of the host-pathogen interaction and its contribution to the development of disease. However, our knowledge concerning the mechanism that Mycobacterium tuberculosis (M. tuberculosis) employs for the entry into the host cell, its survival and multiplication, its spread to neighboring cells and circumventing the host defense mechanisms to cause disease, remains rather poor.

Pathogenicity of a microorganism normally depends on the ability of the organism to survive and replicate in the host. Characterization of virulence determinants is one of the major issues in understanding the pathogenesis of M. tuberculosis. Over years of its evolution M. tuberculosis has developed mechanisms to circumvent the hostile environment of the macrophage. These mechanisms include inhibition of normal phagosome-lysosome fusion (Armstrong & D'Arcy, 1971), selective exclusion of the proton-ATPase system responsible for acidification of phago-lysosomes (Sturgill-Koszycki et al., 1994), and recruitment and retention of the host protein TACO on phagosomes for preventing their delivery to lysosomes (Ferrari et al., 1999). These mechanisms which allow mycobacteria to escape the bactericidal effects of macrophages require live bacteria (Small et al., 1994), suggesting that live bacteria have an ability to trigger specific signals which interfere with the normal functioning of the host cell.

The importance of tyrosine phosphorylation in eukaryotic cells has been established over the past 20 years. Reversible phosphorylation of tyrosine residues has been shown to represent a key mechanism for the transduction of signals that regulate eukaryotic cell growth, differentiation, mobility, metabolism and survival. (Yarden & Ullrich, 1988). The level of phosphorylation on tyrosine residues required for the normal functioning of cells is maintained by the opposing actions of tyrosine kinases and phosphatases (Stone et al., 1994). In recent years, protein phosphorylation in bacteria has been shown to play an important role in sensing extracellular signals and coordinating intracellular events (Kennelly & Potts, 1996). In certain pathogenic bacteria like Yersinia pseudotuberculosis (Galyov et al. 1993; Guan & Dixon, 1990), Salmonella typhimurium (Kaniga et al., 1996) and enteropathogenic E.coli (Rosenshine et al., 1992) tyrosine kinases and phosphatases have been shown to act as major virulence determinants. In Yersinia it has been shown that expression of a tyrosine phosphatase disrupts the host signal transduction processes involved in bacterial uptake and killing (Bliska et al., 1991). In contrast, the mechanisms, which allow mycobacteria to survive in the hostile environment of macrophages, are not understood.

In this application, we report the cloning and characterization of two tyrosine phosphatases from M. tuberculosis. The proteins were expressed in E.coli as GST fusion proteins and characterized by determining their catalytic activity. In addition, we show that these phosphatases are secreted into the culture medium. Based on the knowledge of their functions in other pathogens and their function in normal physiological processes, it is assumed that these tyrosine phosphatases play an important role in the pathogenicity of mycobacteria which may be caused by interference with phosphotyrosine mediated signal transduction processes in macrophages.

In a first aspect the present invention relates to a composition capable of inhibiting or preventing mycobacterial growth, comprising an inhibitor of secretory tyrosine phosphatases from mycobacteria as an active agent.

The inhibitor is a substance which is capable of at least partially inhibiting the biological activity of mycobacterial tyrosine phosphatases, e.g. by inhibiting the interaction of mycobacterial phosphatases with phosphotyrosine mediated signal transduction processes in host cells, e.g. macrophages. The inhibitor may directly interact with the phosphatase or indirectly interact with cellular target molecules of the phosphatase. The inhibitor is preferably a selective inhibitor of microbacterial phosphatases, i.e. a substance, which substantially does not inhibit mammalian tyrosine phosphatases, particularly human tyrosine phosphatases. The inhibitor may be a low molecular weight substance or a high molecular weight biological substance such as an antibody. It should be noted that the term “antibody” includes polyclonal or monoclonal antibodies and any antigen-binding antibody fragment which may be obtained by enzymatic cleavage of an antibody or by genetic engineering. Particularly, this term encompasses genetically engineered antibodies, e.g. chimeric antibodies, humanized antibodies or recombinant single chain antibodies or antibody fragments.

The tyrosine phosphatase inhibitor may act as an inhibitor of mycobacterial growth, particularly as an inhibitor of M. tuberculosis growth.

A further aspect of the present invention is an immunogenic composition comprising (a) a secretory tyrosine phosphatase from mycobacteria or an immunogenic fragment thereof, and/or (b) a nucleic acid encoding a secretory tyrosine phosphatase from mycobacteria or an immunogenic fragment thereof.

The immunogenic composition is capable of eliciting the production of antibodies when administered to a mammal such as an experimental animal or a patient e.g. a human patient. Preferably, the immunogenic composition is a pharmaceutical composition which may comprise a pharmaceutically acceptable carrier and optionally an adjuvant for enhancing the immunogenicity such as Freund's adjuvant, Al₂O₃, cholera toxine etc. More preferably, the composition is a vaccine which is capable of raising protective antibodies when administered to a mammal.

The immunogenic composition may be a polypeptide or peptide vaccine which comprises a mycobacterial tyrosine phosphatase or an immunogenic fragment thereof, wherein said immunogenic fragment preferably has a length of at least six amino acids. The immunogenicity of a peptide fragment may be determined by a molecular analysis of the polypeptide according to the Chou-Fassman model and selecting hydrophilic peptide fragments. Subsequently, the immunogenicity of a given pepide fragment may be experimentally determined according to standard methods by synthesizing the corresponding peptide or polypeptide by means of chemical synthesis or recombinant DNA technology and administering said peptide or polypeptide to an experimental animal and monitoring the immune response.

The immunogenic composition may also comprise a nucleic acid encoding a mycobacterial secretory tyrosine phosphatase or an immunogenic fragment thereof. In this embodiment the composition may be administered to an experimental animal or a patient in a form which allows uptake of the nucleic acid into antigen presenting cells such as macrophages and subsequent expression of the nucleic acid. Thus, the nucleic acid is preferably operatively linked to an expression control sequence which is functional in the target cell. Further, the composition may comprise suitable vehicles, which may enhance transfer to target cells. In this context reference is made to several publications describing mycobacterial DNA vaccines (Lowrie et al., 1999; Lowrie et al., 2000; Tanghe et al., 1999; Baldwin et al., 1999; Kamath et al., 1999; Morris et al., 2000).

The mycobacterial secretory tyrosine phosphatase may be encoded by

(a) a nucleic acid comprising the nucleotide sequence as shown in SEQ ID NO:1 (MptpA) or SEQ ID NO:3 (MptpB) or a nucleic acid complementary thereto,

(b) a nucleic acid corresponding to the sequence of (a) within the scope of degeneracy of the genetic code, i.e. a nucleic acid which differs from the sequence of (a), but encodes the same polypeptide, or

(c) a nucleic acid which hybridizes under stringent condition with a sequence of (a) and/or (b).

More preferably, the secretory tyrosine phosphatase comprises the amino acid sequence as shown in SEQ ID NO:2 (MptpA) or SEQ ID NO:4 (MptpB).

Apart from the nucleotide sequence as shown in SEQ ID NO:1 or SEQ ID NO:3, the present invention also comprises nucleic acid sequences hybridizing therewith under stringent conditions. In the present invention the term “hybridization under stringent conditions” is used as defined in Sambrook et al. (Molecular Cloning. A Laboratory Manual, Cold Spring Harbor Laboratory Press (1989), 1.101-1.104). Thus, a hybridization under stringent conditions takes place, if a positive hybridization signal can still be observed after washing for one hour with 1×SSC and 0.1% SDS at 55° C., preferably at 62° C. and more preferably at 68° C., particularly for one hour in 0.2×SSC and 0.1% SDS at 55° C., preferably at 62° C. and more preferably at 68° C. A sequence hybridizing with a nucleotide sequence as shown in SEQ ID NO:1 or SEQ ID NO:3 under such washing conditions is a mycobacterial tyrosine phosphatase encoding nucleotide sequence according to the present invention.

It should be noted that the nucleic acid sequence of the invention may also encode a fusion polypeptide containing several domains, wherein one of said domains is a mycobacterial tyrosine phosphatase or a fragment thereof and the other domain is a heterologous polypeptide or peptide.

The nucleic acid may be located on a recombinant vector comprising at least one copy of a nucleic acid molecule as defined above. The recombinant vector may be a prokaryotic vector, i.e. a vector containing elements for replication and/or genomic integration in prokaryotic cells. Alternatively, the recombinant vector may be a eukarytotic vector, i.e. a vector containing elements for replication and/or genomic integration in eukaryotic cells, particularly mammalian cells, e.g. human cells. Preferably, the recombinant vector contains the nucleic acid molecule of the present invention operatively linked with an expression control sequence. Examples of such vectors are known to the person skilled in the art and, for instance, illustrated in Sambrook et al., supra.

The composition of the present invention may be used for the manufacture of an agent for the inhibition or prevention of mycobacterial growth. While not wishing to be bound by theory, it is presently assumed that in the course of a mycobacterial infection the tyrosine phosphatases are translocated into the host macrophages thereby modifying the phosphorylation levels of host proteins and as such interfering with the host cell signal transduction pathways. By inhibiting this interference the growth, i.e. survival, proliferation and/or pathogenicity, of mycobacteria may be inhibited. Thus, the compositions of the present invention are suitable for the inhibition or prevention of mycobacterial diseases, particularly of diseases caused by M. tuberculosis. Most preferably, the compositions of the invention are suitable for the treatment or prevention of tuberculosis.

Thus, a method for the inhibition or prevention of mycobacterial growth is provided comprising administering a composition as described above in an effective amount, to a cell or an organism, e.g. a human patient in need thereof, e.g. a subject suffering from a mycobacterial infection or a subject which is in need of a prophylactic administration to avoid the outbreak of a mycobacterial infection.

The compositions of the present invention may contain pharmaceutically acceptable carriers, diluents and auxiliary agents. Further, the compositions may also contain other pharmaceutically active agents, e.g. antibacterial agents such as antibiotics. The pharmaceutical compositions may be suitable for oral, parenteral, e.g. intradermal, intravenous or intramuscular, rectal, nasal and topical applications. The compositions may be injectable solutions, ointments, creams, sprays or aerosols. Further, the compositions may have retardation properties, i.e. showing a delayed release of the active agent.

The dosage of the active agent depends on the specific compound being administered, the type and the severity of the disease. Further, the dosage and the administration protocols will depend on the type of the composition, i.e. if a direct inhibition, i.e. by administering an antibody, or an immunization should be achieved.

Still a further subject matter of the present invention is an antibody against secretory tyrosine phosphatases from mycobacteria. Preferably, the antibody is directed against the MptpA or MptpB tyrosine phosphatases from M. tuberculosis and substantially does not cross-react with other mammalian secretory tyrosine phosphatases. The antibody may be a monoclonal antibody or a polyclonal antibody, e.g. a monospecific polyclonal antibody. Polyclonal antibodies are obtained by immunizing experimental antibodies with a tyrosine phosphatase or an immunogenic fragment thereof and obtaining the antiserum from the immunized experimental animal. For the immunization standard protocols such as described in Harlow and Lane (Antibodies: A Laboratory Manual, Cold Spring Harbor Laboratory Press, Cold Spring Harbor, N.Y.) may be used. Monoclonal antibodies may be obtained from spleen cells of immunized experimental animals according to the method of Köhler and Milstein or subsequent modifications thereof.

Still another embodiment of the present invention relates to a method for the detection of mycobacterial growth comprising contacting a sample suspected to contain mycobacteria or secretory products thereof with a reagent specific for secretory phosphatases from mycobacteria. The sample is usually a biological sample which is obtained from body fluids or tissue of an organism to be tested, e.g. a human patient. The detection of secretory phosphatases may be carried out according to known test formats, e.g. an immunological assay using tyrosine phosphatase specific antibodies. Alternatively, the assay may be a nucleic acid hybridization assay comprising detecting the nucleic acid encoding a mycobacterial tyrosine phosphatase.

Finally, the present invention refers to a method of determining, if a test substance is an inhibitor of mycobacterial growth, comprising determining the effect of the test substance on a secretory phosphatase from mycobacteria. The method may be a so-called cellular assay, wherein the effect of the test substance on a cell expressing, e.g. overexpressing a mycobacterial tyrosine phosphatase is determined. Alternatively, the assay may be a molecular assay, wherein the effect of the test substance on a substantially purified mycobacterial tyrosine phosphatase is determined.

Further, the present shall be explained in more detail according to the following figures and examples:

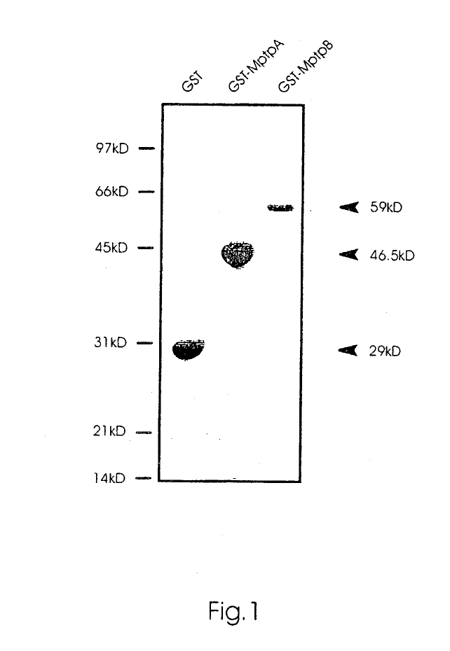

FIG. 1

Electrophoretic Analysis of Recombinant Tyrosine Phosphatases.

Affinity purified tyrosine phosphatases were separated on 12.5% SDS-PAGE and stained with Coomassie Blue. Lane 1, glutathione S-transferase (GST) protein; Lane 2, GST-MptpA fusion protein; and Lane 3, GST-MptpB fusion protein.

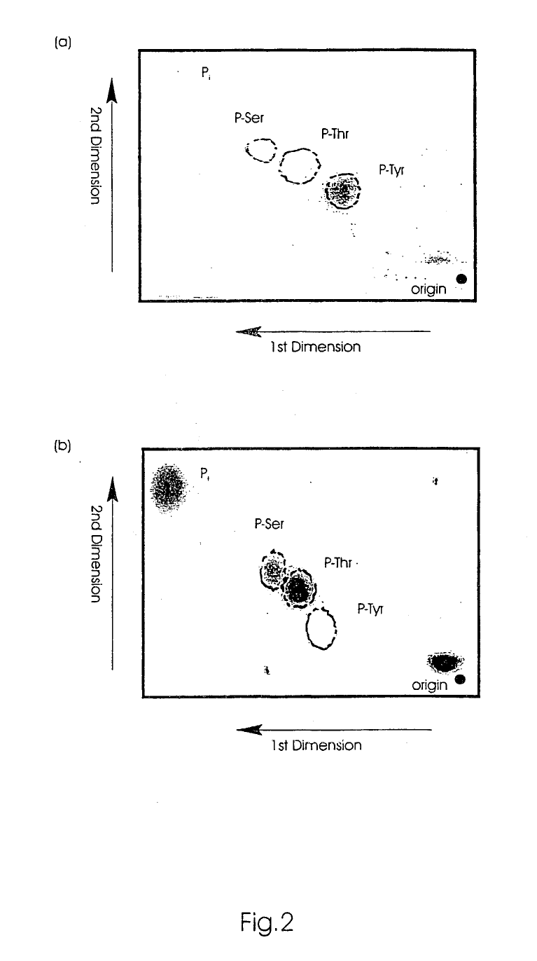

FIG. 2

Analysis of Phosphorylated Residues of mycelin basic protein (MBP).

MBP was phosphorylated by either (A) Src kinase or (B) ERK2 Kinase using γ³²P-ATP. Phosphorylated MBP was run on a 15% SDS-PAGE and electroblotted on a polyvinylidene fluoride membrane. Bands containing proteins were excised and acid hydrolysed in 5.7 M HCI for 90 min. at 110° C. The acid stable phosphoaminoacids liberated on hydrolysis were separated by two-dimensional electrophoresis and autoradiographed. Peptides represent partially hydrolyzed phosphopeptides. ³²pi was produced by partial acid hydrolysis of labeled aminoacids. Samples of nonradioactive phosphotyrosine, phosphoserine, and phosphothreonine were run in parallel and visualized by ninhydrin staining.

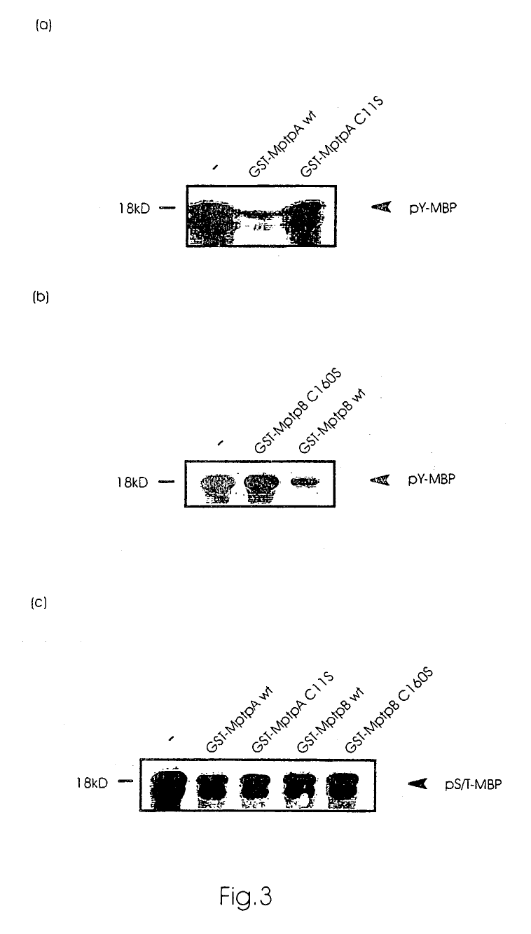

FIG. 3

Protein Dephosphorylation Assays

³²P-Tyr labelled MBP (0.5 μg) or ³²P-Ser/Thr labelled MBP was incubated with purified native and mutant tyrosine phosphatases (0.3 μg) for 120 min at 37° C. The samples were loaded on 15% SDS-PAGE, electroblotted and autoradiographed to determine dephosphorylation. FIG. 3A shows activity of MptpA (Lane 1, MBP alone; Lane 2 and 3 MBP incubated with native or mutant MptpA respectively). FIG. 3B shows activity of MptpB (Lane 1, MBP alone; Lane 2 and 3 MBP incubated with mutant MptpB or native MptpB respectively). FIG. 3C shows activity of MptpA and Mptp B with ³²p labelled Ser/Thr MBP (Lane 1, MBP alone; Lanes 2-5, MBP incubated with native MptpA, mutant MptpA, native MptpB and mutant MptpB respectively).

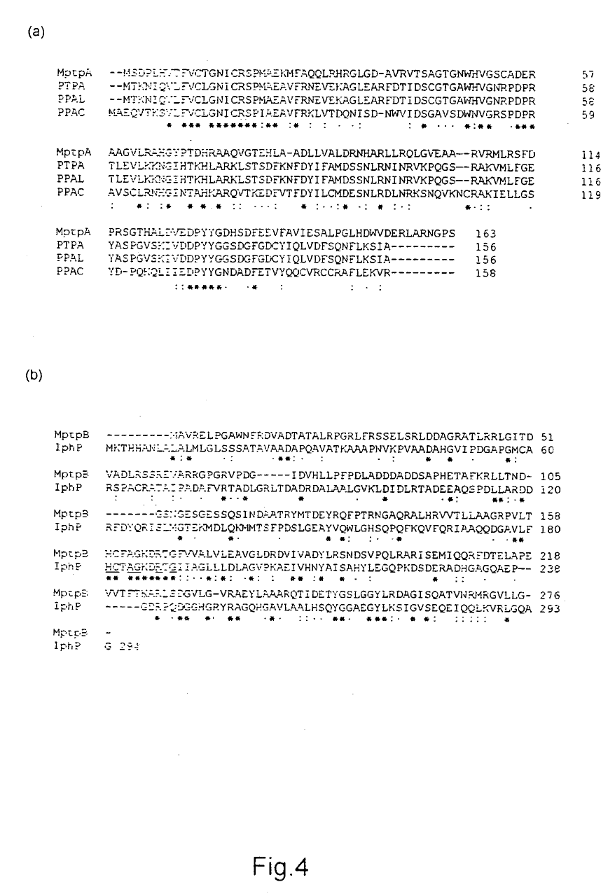

FIG. 4

Comparison of MptpA and MptpB with other known Tyrosine Phosphatases

FIG. 4A shows alignment of MptpA with those of low molecular weight phosphatases from Streptomyces coelicolor (PTPA) (Li & Strohl, 1996); Schizosaccharomyces pombe (PPAL) (Mondesert et al., 1994); PPAC from bovine heart (Wo et al., 1992). FIG. 4B shows alignment of MptpB with Nostoc commune (IphP) (Potts et al., 1993). Identities between catalytic site residues of MptpA and MptpB with other tyrosine phosphatases are shown by boxes. As can be seen in the FIG. 4A the catalytic site domain of MptpA is located at few amino acids downstream from the N-terminus.

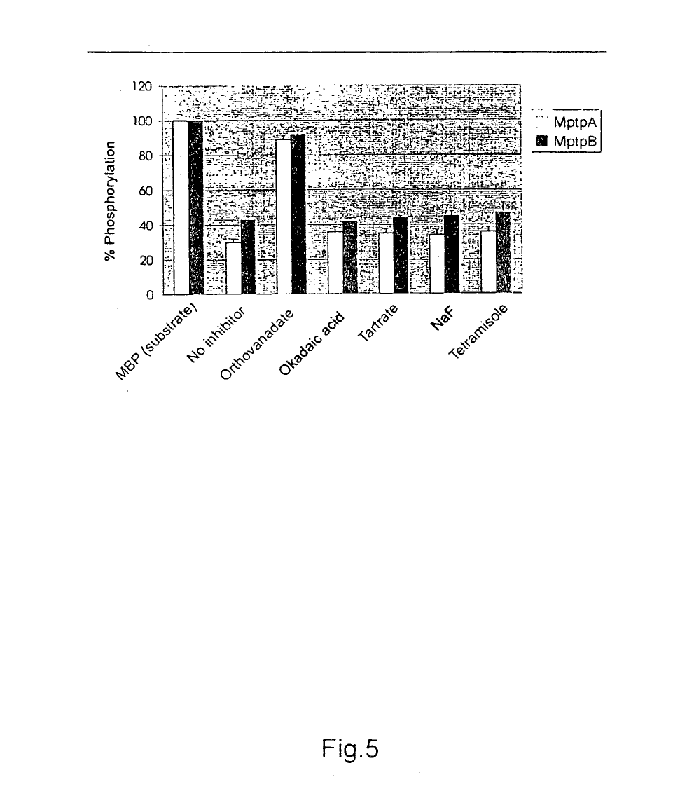

FIG. 5

Effect of Various Inhibitors on the Activity of MptpA and MptpB

³²P-Tyr labeled MBP (0.5 μg) was incubated with MptpA (0.2 μg) in imidazole buffer (pH 7.0) or Mptp B (0.2, μg) in sodium acetate buffer (pH 5.6) containing various inhibitors for 30 min at 30° C. Samples were loaded on 15% SDS-PAGE, electroblotted and dephosphorylation was quantitated using a phosphoimager (Fugi). The values written in parentheses indicate the concentration of the inhibitors. Activity is reported as the percentage of that observed in absence of inhibitor.

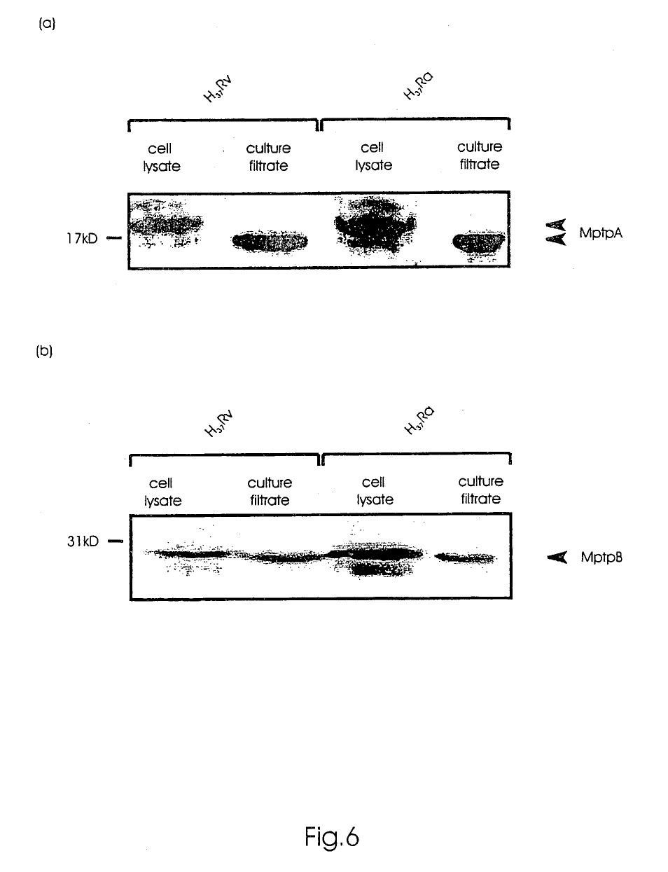

FIG. 6

Expression of Tyrosine Phosphatases in M. tuberculosis

Equal amount of whole cell lysates (40 μg) and culture filtrate proteins (40 μg) from M. tuberculosis strains H₃₇Rv and H₃₇ Ra were loaded on a 15% SDS-PAGE, electroblotted. Blots were probed with anti MptpA (A) or MptpB (B) antibodies and developed using ECL kit (NEN).

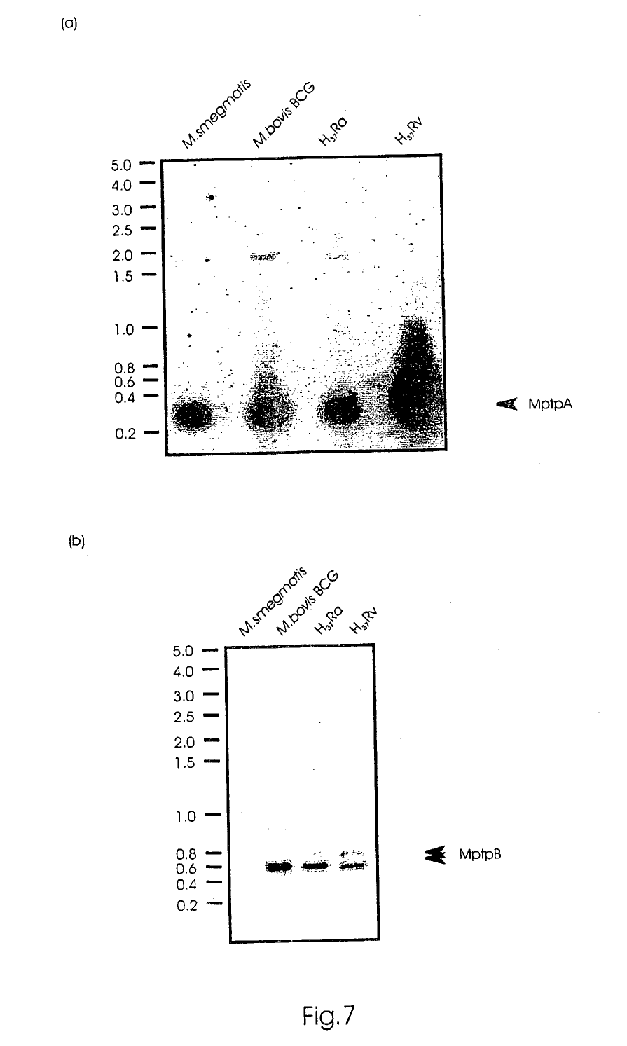

FIG. 7

Presence of Tyrosine Phosphatase Genes in other Mycobacteria

Genomic DNA (7 μg each) from various strain of M. tuberculosis H₃₇Rv, H₃₇Ra, M. bovis BCG and M. smegmatis were digested with restriction enzymes, resolved on a 1% agarose gel at 25-30 V for 16 hrs and transferred to nitrocellulose membranes. The hybridization was performed using a ³²p labeled MptpA (FIG. 7A) and MptpB (FIG. 7B) probe and autoradiographed. Lane 1, M. smegmatis; Lane 2, BCG; Lane 3, H₃₇Ra; and Lane 4, H₃₇Rv.

### EXAMPLES

1. Materials and methods

1.1 Bacterial Strains, Plasmids and Antibodies

Whole cell lysates and culture filtrate proteins of M. tuberculosis (H₃₇Rv and H₃₇Ra) were provided by Dr. John T. Belisle (Colorado, USA) under the “TB research material and vaccine testing program” of NIH, NIAID (contract no Al—75320). Genomic DNA of M. tuberculosis H₃₇Rv and H₃₇Ra, M. bovis BCG and M. smegmatis were provided by Dr. K. Drlica from The Public Health Research Institute, N.Y., USA. The expression plasmid (pGEX-5X-3) used for the expression was purchased from Pharmacia. Rabbit polyclonal antisera against ERK2 were purchased from Santa Cruz Biotechnology, USA and anti-Src antibodies (mouse monoclonal) were obtained from Upstate Biotechnology, USA.

1.2 Plasmid Construction and mutagenesis

M. tuberculosis H₃₇Rv genomic DNA was used as a template for amplification of two putative tyrosine phosphatase genes by polymerase chain reaction (PCR) (Cole et al., 1998). The two genes were designated MptpA (492 bp) and MptpB (831 bp). The sequence of the two PCR primers for cloning MptpA were:

5′-GGAATTCCATGTCTGATCCGCTGCACGTCACATTC-3′ for the 5′ end (carrying an EcoRI site) and

5′ CCGCTCGAGTCAACTCGGTCCGTTCCGCGCGAGAC-3′ for the 3′ end of the gene (carrying XhoI site).

To clone the MptpB gene the sequence of the two primers were: 5′- CGGGATCCCGATGGCTGTCCGTGAACTGCCGGG-3′ for the 5′ end of the gene (containing BamHl site) and

5′-CGAATTCTCATCCGAGCAGCACCCCGCGCATCCG-3′ for the 3′ end of the gene (containing an EcoRI site).

PCR amplification was carried out using a standard protocol. The amplified product of MptpA gene was digested with EcoRI and XhoI and ligated into pGEX-5X-3 plasmid which was previously digested with the same restriction enzymes and the resulting plasmid was designated as pGEX-MptpA. Similarly, the PCR amplified product of MptpB gene was digested with BamH1 and EcoR1 and ligated with the BamHI and EcoRI digested pGEX-5X-3 plasmid. The resulting plasmid was designated as pGEX-MptpB. Site directed mutagenesis of cysteine 11 of MptpA and cysteine 160 of MptpB genes to serine residues was carried as described previously (Kunkel et al., 1991). The oligonucleotide for mutating cysteine 11 to serine in the MptpA gene was:

5′-GTCACATTCGTTAGTACGGGCAACATC-3′ and the oligonucleotide for mutating cysteine 160 to serine in MptpB gene was:

5′-CCGGTGCTCACCCACAGCTT CGCGGGTAAGGATC-3′ (the underlined bases indicate the change from cysteine to serine). The plasmids with the mutant genes were designated as pGEX-MptpA-C11S and pGEX-MPtpB-C160S for MptpA and MptpB, respectively. The nucleotide sequence of each gene was determined by sequencing using the dideoxynucleotide method (Sanger et al., 1977). The nucleotide sequence for the MptpA gene and the amino acid sequence of the corresponding polypeptide is shown in SEQ ID NO:1 and 2. The nucleotide sequence for the MptpB gene and the amino acid sequence of the corresponding polypeptide is shown in SEQ ID NO:3 and 4.

1.3 Expression and Purification of MptpA and MptpB

Escherichia coli (E. coli) BL21 was separately transformed with pGEX-MptpA or pGEX-MptpB, pGEX-MptpA-C11S, and pGEX-MptpB-C160S plasmids. Transformants were grown in 2YT medium containing 100 μg/ml ampicillin at 37° C. until the A₆₀₀ reached 0.5. Isopropyl-1-thio-β-D-galactopyranoside (IPTG) was then added to a final concentration of 0.5 mM and cultures were further grown for 5 hrs at 37° C. with shaking. Cells were harvested by centrifugation at 5,000×g for 15 min and suspended in 20 ml of sonication buffer (50 mM Tris-CI pH 7.4, 150 mM NaCl, 1 mM EDTA, 10% glycerol, 1 mM phenylmethylsulfonyl fluoride and 10 μg/ml aprotinin). The cells were then sonicated on ice for 2 minutes and the sonicate was supplemented with Triton X-100 to a final concentration of 1% before centrifugation at 30,000×g for 30 min. at 4° C. The supernatant was incubated overnight at 4° C. with a glutathione-Sepharose 4B matrix (Pharmacia Biotech). The resin bound to protein was packed into a column and washed with five bed volumes of phosphate buffered saline (PBS). Protein was eluted with 50 mM Tris-Cl, pH 8.0 containing 1 mM DTT, 5 mM MgCl₂ and 15 mM glutathione. Fractions were analyzed by 15% sodium dodecyl sulfate polyacrylamide gel electrophoresis (SDS-PAGE) (Laemmli, 1970). Fractions containing purified fusion proteins were pooled and dialyzed against PBS containing 20% glycerol and stored at −20° C. By using this procedure both tyrosine phosphatases (MptpA and MptpB) as well as their mutants were purified.

1.4 Preparation of ³²P-labelled Phosphoprotein Substrate

Human 293 embryonic kidney cells (ATCC CRL-1573) were grown in Dulbeccos Modified Eagles Medium supplemented with 2 mM glutamine and 10% fetal calf serum. The cells were then transfected separately with plasmid p60^{c-Src} carrying the Src kinase (tyrosine kinase) or a plasmid carrying the ERK2 kinase gene (serine/threonine kinase) as described (Chen & Okayama, 1987). The cells overexpressing the desired proteins were lysed in lysis buffer and Src kinase and ERK2 kinase were immunoprecipitated from the cell lysates using the anti-Src or anti-ERK2 antibodies as described (Zwick et al., 1999). The immunoprecipitate was washed three times with 0.5 ml of washing buffer (20 mM Hepes, pH 7.5, 150 mM NaCl, 1% Triton X-100, 10% glycerol, 10 mM NaF and 1 mM sodium orthovanadate) and washed once with kinase buffer (20 mM Hepes, pH 7.5, 10 mM MgCl₂₁ 1 mM DTT and 200 μM sodium orthovanadate).

The substrate myelin basic protein (MBP) was phosphorylated either at tyrosine residues by immunoprecipitated Src kinase or at serine/threonine residues using ERK2 kinase in separate reactions. In brief, MBP (10 μg) was incubated at 30° C. for 30 min. with kinase in the kinase buffer (20 μl) containing 20 μCi (γ−³²P)-ATP. The reaction was stopped and unincorporated ATP was removed by adding ice cold trichloroacetic acid (TCA) (25% final concentration). The precipitate was washed twice with 10% TCA and once with acetone. The phosphorylated substrates were dissolved in 25 mM imidazole, pH 7.4 and used for dephosphorylation assays. The phosphorylated substrates were analysed for phoshorylated amino acids as described earlier (Vincent et al., 1999).

1.5 Phosphatase Assay

The phosphatase assay is based on the measurement of release of ³²pi from ³²P-labelled substrates. The activity of purified MptpA or its mutant derivative was assayed by incubating phosphorylated MBP (0.5 μg) for 120 min at 37° C. in an imidazole buffer (25 mM, pH 7.0) containing 0.05% β-mercaptoethanol and 0.1 mg/ml BSA. Similarly, the activity of MptpB and its mutant protein was determined by using sodium acetate (50 mM, pH 5.6). The reactions were terminated by the addition of SDS sample buffer and analysed on 15% SDS-PAGE. The gel was electroblotted to a nitrocellulose membrane and autoradiographed to determine dephosphorylation.

1.6 Purification of Rabbit Antibodies against MptpA and MptpB

Purified GST-MptpA fusion protein (500 μg) and GST-MptpB fusion protein (200 μg) were separately solubilised in 1 ml Complete Freund's adjuvant and injected into rabbits. Subsequently, three injections of 250 pg each in 1 ml of incomplete Freund's adjuvant were given after an interval of 15 days. Ten days after the final injection, animals were bled and titers of anti GST-MptpA and anti GST-MptpB were determined by ELISA as described (Harlow & Lane, 1988). The antibodies specific to MptpA and MptpB were isolated by passing the serum on sepharose resin coupled to either MptpA or MptpB. The coupling of sepharose to phosphatases and purification of antibodies were performed by following the standard method as described earlier (Harlow & Lane, 1988). The purified antibodies specific to MptpA and MptpB were used to study the expression of tyrosine phosphatases of M. tuberculosis.

1.7 Analysis of Mycobacterial Tyrosine Phosphatases

Equal amount of protein from whole cell lysates and culture filtrates of M. tuberculosis strains H₃₇Rv and H₃₇ Ra were loaded on a 15% SDS-PAGE and transferred to nitrocellulose membrane. The blots were probed with purified rabbit anti-MptpA and anti-MptpB antibodies and developed with ECL kit (NEN).

1.8 Southern Blot Analysis

Genomic DNA (7 μg each) from M. tuberculosis H₃₇Rv, H₃₇Ra, M. bovis BCG and M. smegmatis were digested with restriction enzymes (Hinc II and Xmn I for MptpA and Hinc II and Xma I for MptpB genes). Digested products were run on a 1% agarose gel at 25-30 V for 16 hrs and transferred to nitrocellulose membranes. The hybridization was performed at 66° C. using 6×SSC (1×SSC is 150 mM sodium chloride and 15 mM sodium citrate, pH 7.2) using ³²p labeled MptpA and MptpB probe as described earlier (Reyrat et al., 1995) and subjected to autoradiography.

2. Results

2.1 Expression and Purification of MptpA and MptpB

The complete sequence of M. tuberculosis genome (Cole et al., 1998) has revealed two DNA sequences which encode translation products of 17.5 kDa (MptpA) and 30 kDa (MptpB). Both of these genes were amplified by PCR using oligonucleotide primers deduced from the genome sequence of M. tuberculosis (Cole et al., 1998). The amplified DNA products of MptpA and MptpB gene were cloned in EcoR1-Xho1 and BamH1-EcoR1 sites of pGEX-5X-3, respectively. The resulting plasmids (pGEX-MptpA and pGEX-MptpB) were used to transform E.coli and the transformants expressed fusion proteins of MptpA and MptpB with glutathione-S-Transferase (GST, 29 kDa) at its NH₂-terminal. An in vitro transcription and translation assay was carried out in order to confirm that pGEX-MptpA and pGEX-MptpB encoded translation products of 46.5 kDa (GST+MptpA) and 59 kDa (GST+MptpB), respectively.

The expressed GST-fusion proteins (GST-MptpA or GST-MptpA) were purified using a glutathione-Sepharose 4B matrix. The purified fusion proteins were analyzed by SDS-PAGE (FIG. 1) and the size of the fusion proteins was found to be consistent with the calculated molecular mass of these proteins. The typical yield of purified proteins was about 2 mg from 1 liter of bacterial culture.

2.2 Phosphotyrosine Activity of MptpA and MptpB

The tyrosine phosphatase activity of the purified proteins was determined by their ability to dephosphorylate tyrosine phosphorylated Myelin Basic Protein (MBP). A phosphoaminoacid analysis of MBP was performed to identify specific phosphorylated residues of the substrate. Labeled MBP was acid hydrolyzed and analyzed by two dimensional thin layer chromatography. Incubation of MBP with immunoprecipitated Src kinase led to the phosphorylation of tyrosine residues (FIG. 2A) alone whereas, MBP incubated with immunoprecipated ERK2 phosphorylated serine/threonine residues (FIG. 2B). Incubation of purified MptpA with tyrosine phosphorylated MBP dephosphorylated tyrosine residues efficiently at pH 7.0 (FIG. 3A). Similarly, MptpB dephosphorylated tyrosine residues of phosphorylated MBP (FIG. 3B). The optimum dephosphorylation of tyrosine residues of MBP by MptpB was observed at pH 5.5-5.8.

MptpB showed 26.8% sequence homology to tyrosine/serine phosphatase (IphP) of Nostoc commune (Potts et al., 1993). This phosphatase has been shown to display phosphatase activity towards both tyrosine and serine residues. Thus, the substrate specificity of purified MptpA and MptpB was determined using MBP substrate phosphorylated at serine/threonine residues. Both MptpA and MptpB did not dephosphorylate serine/threonine residues of MBP unlike lphP (FIG. 3C) suggesting that mycobacterial phosphatases are specific for tyrosine residues.

2.3 Role of Catalytic Cysteines of MptpA and MptpB

MptpA is a low molecular weight phosphatase and the sequence homology of the catalytic domain of MptpA with the catalytic domains of other low molecular weight phosphatases revealed a striking similarity (FIG. 4A). The conserved catalytic site cysteine of low molecular weight phosphatases has been shown to be essential for their activity (Grangeasse et al., 1998). In order to determine the role of cysteine 11, present in the catalytic domain of MptpA, it was mutated to serine. The mutant protein (GST-MptpA-C11S) was expressed, purified and assayed for activity. Consistent with the properties of other protein tyrosine phosphatases the mutant protein had no enzymatic activity suggesting that Cysteine 11 is crucial for the enzymatic activity (FIG. 3A).

Similarly, comparison of the catalytic sites of MptpB with other bacterial and eukaryotic tyrosine phosphatases revealed a similarity in the amino acid sequences (FIG. 4B). The catalytic site Cysteine (Cys 160) of MptpB was substituted with Serine by site directed mutagenesis and the mutant protein (GST-MptpB -C160S) was expressed and purified. The mutant protein failed to dephosphorylate tyrosine phosphorylated MBP indicating loss of enzymatic activity (FIG. 3B).

2.4 Inhibition of Enzymatic Activities of MptpA and MptpB

The activities of MptpA and MptpB towards tyrosine phosphorylated MBP were inhibited by sodium orthovanadate, an inhibitor of protein tyrosine phosphatases. However, okadaic acid, a potent inhibitor of protein serine/threonine phosphatases, tetramisole, an inhibitor of alkaline phosphatase, tartrate, an acid phosphatase inhibitor and sodium fluoride, a nonspecific inhibitor for serine/threonine phosphatases had no effect on the activity of MptpA or MptpB (FIG. 5).

2.5 Western Blot Analysis of Mycobacterial Tyrosine Phosphatases

Monospecific polyclonal antibodies raised against MptpA and MptpB were used to analyze the expression of tyrosine phosphatases of growing mycobacterial cultures. Equal amounts of mycobacterial whole cell lysates and culture filtrate proteins from M. tuberculosis H₃₇Rv and H₃₇Ra strains were separated on a 15% SDS-PAGE and electroblotted on nitrocellulose membrane. The membranes were incubated with monospecific antibodies and visualized using ECL kit (NEN). Both MptpA and MptpB were present in whole cell lysates of M. tuberculosis H₃₇Rv and H₃Ra. The culture filtrate, which was prepared from the mid log phase growing mycobacterial cells, also showed the presence of MptpA and MptpB proteins, suggesting that these phosphatases are secreted into the culture medium by growing mycobacteria cells (FIGS. 6A and B).

2.6 Analysis of Prevalence of Tyrosine Phosphatases in other Species of Mycobacteria

The PCR products of MptpA (492 bp) and MptpB (831 bp) genes were used in southern hybridization experiments to determine the prevalence of MptpA and MptpB homologs in various species of mycobacteria. Hybridization results revealed that a MptpA homologous gene was present in all the members of M. tuberculosis complex analyzed in this study as well as M. smegmatis—a saprophyte. However, MptpB homologous gene sequences were found to be present exclusively among the members of M. tuberculosis complex analysed in this study. The gene was found to be absent in the case of M. smegmatis—a non-pathogenic species of mycobacteria. (FIGS. 7A & B).

3. Discussion

Protein tyrosine phosphatases (PTPs) have long been considered to be confined to eukaryotes. It is only in recent times that genes encoding protein tyrosine phosphatases have been found in some bacterial species (Kennelly & Potts, 1996; Li & Strohl, 1996). Protein tyrosine phosphatases have been shown to be involved in the pathogenicity of several prokaryotes. Yersinia pseudotuberculosis secretes a protein tyrosine phosphatase (YopH) which is essential for the survival of Yersinia in the host cells (Guan & Dixon, 1990). YopH is secreted into the extracellular medium by the bacterium and is targeted to the inner surface of macrophages where it dephosphorylates certain host proteins which are implicated in the bactericidal action (Bliska et al., 1991; Black & Bliska, 1997). In Salmonella typhimurium, an intracellular pathogen, a protein tyrosine phosphatase (SptP) has been shown to play a critical role in the pathogenesis of this bacterium (Kaniga et al., 1996; Fu & Galan, 1999). The entry and survival of intracellular pathogens into the host cells requires a complete dialogue of signaling events between the host cells and the pathogenic bacteria mediated by certain unique regulatory molecules like protein tyrosine phosphatases and kinases (Galan & Bliska, 1996). Therefore, understanding the mechanisms involved in the signal cross talk between the bacterial pathogens and their host cells may help us in the development of effective therapeutic targets against these diseases.

M.tuberculosis is an intracellular pathogen and has developed successful strategies to invade and replicate within the macrophages. The entry of M. tuberculosis into macrophages and subsequent events appear to involve specific signals between the host cell and the bacterium suggesting that secreted molecules may be necessary for the reprogramming of the host signaling network which may help the bacterium in its propagation causing at the same time pathogenic defects. Thus we decided to characterize the PTPs from mycobacteria in order to evaluate their role in the pathogenesis of M. tuberculosis. In this study two genes with sequence homology to protein tyrosine phosphatases were cloned from the genome DNA sequence of M. tuberculosis (Cole et al., 1998). The putative PTP DNA sequences were expressed in Ecoli and upon affinity purification of these proteins they were characterized for their specificity by several methods. The PTP genes of M. tuberculosis encoded 17.5 kDa (MptpA) and 30 kDa (MptpB) proteins and contained a characteristic catalytic domain matching that of previously identified protein tyrosine phosphatases (Stone & Dixon, 1994).

It has been observed that several protein tyrosine phosphatases like IphP in Nostoc commune and Stp 1 in Schizosaccharomyces pombe can dephosphorylate both tyrosine as well as serine/threonine residues of their substrates (Potts et al., 1993; Zhang et al., 1995). In order to determine the substrate specificity of the PTPs from M. tuberculosis, MBP phosphorylated either at tyrosine or serine/threonine residues was used as a substrate in a dephosphorylation reaction with purified MptpA or MptpB protein. Both MptpA and MptpB were specific for phosphotyrosine residues of the MBP without showing any activity for phosphoserine or phosphothreonine residues.

MptpA displays sequence homology with other known low molecular weight tyrosine phosphatases isolated from bovine heart and yeast Schizosaccharomyces pombe (Wo et al., 1992 and Mondesert et al., 1994). Low Molecular Weight (LMW) phosphatases (previously called acid phosphatases) have only a catalytic domain without any regulatory domains unlike other tyrosine phosphatases which contain both catalytic as well as regulatory domains (Fauman & Saper, 1996). The catalytic domain of LMW phosphatases is present at the few amino acids from the N-terminal of the protein. Site directed mutagenesis of cysteine 11 to serine in MptpA completely abolishes its enzymatic activity suggesting that cysteine 11 is the conserved catalytic site residue and the catalytic domain is present adjacent to the N-terminus of the protein as in the case of other LMW phosphatases. MptpB exhibits sequence homology with the protein tyrosine phosphatase (IphP) of Nostoc commune whose catalytic site, unlike LMW phosphatases, is located towards the C-terminal portion of the protein (Potts et al., 1993). This sequence homology between MptpB and lphP suggests an evolutionary connection between mycobacterial and other prokaryotic tyrosine phosphatases. The catalytic cysteine is highly conserved in all protein tyrosine phosphatases and is required for the formation of covalent phosphoenzyme intermediate (Chiarugi et al., 1992). On mutating cysteine 11 of MptpA and cysteine 160 of MptpB to serine, we found that these mutant proteins failed to dephosphorylate tyrosine phosphorylated MBP. This demonstrates that cysteine 11 of MptpA and cysteine 160 of MptpB are required for enzyme activity. Our results suggest that the same catalytic mechanism for MptpA and MptpB may exist as employed by other protein tyrosine phosphatases. Further, characterization MptpA and MptpB enzymatic activities, using okadaic acid (an inhibitor for protein serine/threonine phosphatases 1 and 2A), did not inhibit activities of either MptpA or MptpB. Both MptpA and MptpB were also insensitive to tetramisole or tartrate, indicating that their enzymatic activities were not due to contaminating E.coli alkaline or acid phosphatases and suggesting that mycobacterial tyrosine phosphatases are specific for phosphotyrosine residues.

M. tuberculosis is known to secrete a large number of proteins into the extracellular medium. These secreted proteins have been shown to play an important role in the interaction of mycobacteria with the host cell (Harth et al., 1994) and are thought to be the prime candidate molecules for the development of subunit vaccines and new antimycobacterial drugs (Belisle et al., 1997). Both tyrosine phosphatases MptpA and MptpB of mycobacteria were secreted in the culture medium as studied by western blot using specific antibodies. In most of the cases the proteins to be secreted out of the cell have an N-terminal sequence encoding a signal peptide which is responsible for the transport of the proteins outside the cell. The export of these proteins occurs after cleavage of the signal peptides by a specific peptidase. However, in the case of MptpA the presence of the catalytic domain a few amino acids upstream from the N-terminus suggests that this phosphatase lacks a secretory signal peptide. Furthermore comparison of known signal sequences of mycobacterial proteins and conserved signal sequences of tyrosine phosphatases with both MptpA and MptpB by BLAST search provided no indication for the presence of a signal peptide. Nevertheless both tyrosine phosphatases were surprisingly secreted in the extracellular medium by mycobacterial cells growing in mid log phase. Since both MptpA and MptpB lack a signal peptide, the mechanism employed by the mycobacteria in exporting these proteins is not understood.

The gene coding for MptpA was present in the members of M. tuberculosis complex analyzed in this study as well as in M. smegmatis. However, it is interesting to note that the gene coding for MptpB although present in members of the M. tuberculosis complex analyzed in this study, was absent in fast growing, avirulent M. smegmatis which suggests a role in processes which are may be specific to the members of the M. tuberculosis complex.

Thus, the findings of the present study show that mycobacteria express two active tyrosine phoshatases, which are secreted into the culture medium. It is assumed that these phosphatases are translocated into the host macrophages thereby modifying the phosphorylation levels of host proteins and as such interfering with the host cell signal transduction pathways that may be essential for the survival of mycobacteria in macrophages and in its pathogenicity.

### REFERENCES

1. Armstrong, J. & D'Arcy H. P. (1971). Response of cultured macrophages to Mycobacterium tuberculosis, with observation on fusion of lysosomes with phagosomes. J. Exp. Med. 134, 713-740.

2. Belisle, J. T., Vissa, V. D., Sievert, T., Takayama, K., Brennan, P. J. & Besra, G. S. (1997). Role of the major antigen of Mycobacterium tuberculosis in cell wall Biogenesis. Science, 276. 1420-2.

3. Black, D. S. & Bliska, J. B. (1997). Identification of p130cas as a substrate of Yersinia YopH (Yop 51), a bacterial protein tyrosine phosphatases that translocates into mammalian cells and targets focal adhesions. EMBO J. 16, 2730-2744.

4. Bliska, J. B., Guan, K., Dixon, J. E. & Falkow, S. (1991). Tyrosine phosphatase hydrolysis of host proteins by an essential Yersinia virulence determinant. Proc. Natl. Acad. Sci. USA, 88, 1187-1191.

5. Chen, C. & Okayama, H. (1987). High-efficiency transformation of mammalian cells by plasmid DNA. Mol. Cell Biol. 8, 2745-52.

6. Chiarugi, P., Marzocchini, R., Raugei, G., Pazzagli, C., Berti, A., Camici, G., Manao, G., Cappugi, G. & Ramponi, G. (1992). Differential roles of four cysteines on the activity of a low-molecular phosphotyrosine protein phosphatase. FEBS Lett. 310, 9-12.

7. Cole, S. T., Brosch, R., Parkhill, J., Garnier,T., Churcher, C., Harris, D., Gordon, S. V., Eiglmeier, K., Gas, S., Barryl 111, C.E., Tekaia, F., Badcock, K., Basham, D., Brown, D., Chillingworth, T., Connor, R., Davies, R., Devlin, K., Feltwell, T., Gentles, S., Hamlin, N., Holroyd, S., Hornsby, T., Jagels, K., Krogh, A., McLean, J., Moule, S., Murphy, L., Oliver, K., Osborne, J., Quail. M. A., Rajandream, M. A., Rogers, J., Rutter, S., Seeger, K., Skelton, J., Squares, R., Squares, S., SuIston, J. E., Talyor, K., Whitehead, S. & Barrell, B. G. (1998). Deciphering the biology of Mycobacterium tuberculosis from the complete genome sequence. Nature, 393, 537-544.

8. Fauman, E. B. & Saper, M. A. (1996). Structure and function of protein tyrosine phosphatases. TIBS, 21, 413-417.

9. Ferrari, G., Langen, H., Naito, M. & Pieters, J. (1999). A coat protein on phagosomes involved in the intracellular survival of mycobacteria. Cell, 97, 435-447.

10. Fu, Y., Galan, J. E. (1999). A Salmonella protein antagonizes Rac1 and Cdc42 to mediate host-cell recovery after bacterial invasion. Nature 401, 293-297.

11. Galan, J. E. & Bliska, J. B. (1996). Cross talk between bacterial pathogens and their host cells. Annu. Rev. Cell Dev. Bio. 12, 221-51.

12. Galyov, E. E., Hakaansson, S., Forsberg, A. & Wolf, H. W. (1993). A secreted protein kinase of Yersinia pseudotuberculosis is an indispensable virulent determinant. Nature, 859, 588-589.

13. Grangeasse, C., Doublet, P., Vincent, C., Vaganay, E., Riberty, M., Duclos, B. & Cozzone, A. J.(1998). Functional characterization of low-molecular mass phosphotyrosine protein phosphatase of Acinetobacter johnsonii. J. Mol. Biol. 278, 339-347.

14. Gual, K. & Dixon, J. E. (1990). Protein tyrosine phosphatase activity of an essential virulence determinants in Yersinia. Science, 249, 553-556.

15. Harlow, E. & Lane, E. (1988). In Antibodies: A laboratory (Ed. Harlow, E. & Lane, E.). Cold Spring Harbor Laboratory Press, Cold Spring Harbor, N.Y.

16. Harth, G. & Horwitz, M. (1999). Export of recombinant Mycobacterium tuberculosis superoxide dismutase is dependent upon both information in the protein and mycobacterial export machinery. J. Biol. Chem. 274,4281-4292.

17. Harth, G., Clemens, D. L. & Horwitz, M. A. (1994). Glutamine Synthetase of Mycobacterium tuberculosis: extracellular release and characterization of its enzymatic activity. Proc. Natl. Acad. Sci. USA, 91, 9342-6.

18. Kaniga, K., Uralil, J., Bliska, J. B. & Galan, J. E. (1996). A secreted protein tyrosine phosphatase with modular effectors domains in bacterial pathogen Salmonella tyrhimurium. Mol. Microbio. 21, 633-41.

19. Kennelly, P. J. & Potts, M. (1996). Fancy meeting you here! A fresh look at prokaryotic protein phosphorylation. J. Bacteriol. 178, 4759-4764.

20. Kunkel, T. A., Bebenek, K. & McClary, J. (1991). Efficient site-directed mutagenesis using uracil-containing DNA. Methods Enzymol. 204, 125-39.

21. Laemmli, U. K. (1970). Cleavage of structure proteins during the assembly of the head of bacteria phage T4. Nature, 227, 680-685.

22. Li, Y. & Strohl, W. R. (1996). Cloning, purification, and properties of a Phosphotyrosine protein phosphatase from Streptomyces coelicolorA3(2). J. Bacteriol. 178, 136-142.

23. Mondesert, 0., Moreno, S. and Russell, P. (1994). Low molecular weight protein tyrosine phosphatases are highly conserved between fission yeast and man. J. Biol. Chem. 269 (45), 27996-27999.

24. Potts, M., Sun, H., Mockaitis, K., Kennelly, P. J., Reed, D. & Tonks, N. K. (1993). A protein tyrosine/serine phosphatase encoded by the genome of Cyanobacterium Nostoc commune UTEX 584*. J. Biol. Chem. 268, 7632-7635.

25. Reyrat, J. M., Berthet, F. X. & Gicouel, B. (1995). The Urease locus of Mycobacterium tuberculosis and its utilization for the demonstration of allelic exchange in Mycobacterium bovis bacillus Calmette-Guerin. Proc. Natl. Acad. Sci. USA, 92, 8768-8772.

26. Rosenshine, l.,Donnenberg, M. S., Kaper, J. B. & Finlay, B. B. (1992). Signal transduction between enteropathogenic Escherichia (EPEC) and epithelial cells: EPEC induces tyrosine phosphorylation of host cell proteins to initiate cytoskeletal rearrangement and bacterial uptake. EMBO J. 11, 3551-3560.

27. Sanger, F., Nicklen, S. & Coulson, A. R. (1977). DNA sequencing with chain-terminating inhibitors. Proc. Natl. Acad. Sci. USA, 74. 5463-7

28. Small, P. L. C., Ramakrishnan, L. & Falkow, S. (1994). Remodeling schemes of intracellular pathogens. Science, 263, 637-639.

29. Snider, D. E. Jr., Raviglione, M. & Kochi, A. (1994). In Tuberculosis: pathogenesis, protection, and control. (Ed. Bloom, B. R., Am. Soc. Microbiol., Washington DC), pp. 2-11.

30. Stone, R. L. & Dixon, J. E. (1994). Protein tyrosine phophatases. J. Biol. Chem. 269, 31323-31326.

31. Sturgill-Koszycki, S., Sclesinger, P. H., Chakraborty, P., Haddix, P. L., Collins, H. L., Fok, A. K., Allen, R. D., Gluck, S. L., Heuser, J. & Russel, D. G. (1994). Lack of acidification in Mycobacterium phagosomes produced by exclusion of the vascular proton —ATPase. Science, 263, 678-681.

32. Vincent, C., Doublet, P., Grangeasse, C., Vaganay, E., Cozzon, A. Z. & Duclos, B. (1999). Cells of Escherichia coli contain a protein tyrosine kinase, Wzc and a phosphotyrosine-protein phosphatase, Wzb. J. Bacteriol. 181 , 3472-3477.

33. Wattiau, P., Bernier B., Deslee, P., Michiels, T. & Cornelis, G. R. (1994). Individual chaperons required for Yop secretion by Yersinia. Proc. Natl. Acad. Sci.USA, 91, 10493-10497

34. Wo, Y. Y., Zhou, M. M., Stevis, P., Davis, J. P., Zhang, Z. Y. and Van Etten, R. L. (1992). Cloning, expression and catalytic mechanism of the low molecular weight phosphotyrosyl protein phosphatase from bovine heart. Biochemistry, 31, 1712-1721.

35. Yarden, Y. & Ullrich, A. (1988). Growth factor receptor tyrosine kinases. Annu. Rev. Biochem. 57, 443-478.

36. Zhang, Z. Y. & Van Etten, R. L. (1990). Purification and characterization of a low-molecular weight acid phosphatase, a phosphotyrosyl-protein phosphatase from bovine heart. Arch. Biochem. Biophys. 282, 39-49.

37. Zhang, Z. Y., Zhou, G., Denu, J. M., Wu, L., Tang, X., Mondesert, O., Russel, P., Butch, E. & Guan, K. L. (1995). Purification and characterization of the low-molecular weight tyrosine phosphatase Stp1 from the fission yeast Schizosaccaromyces pombe. Biochemistry, 34, 10560-10568.

38. Zwick, E., Wallasch, C., Daub, H. & Ullrich, A. (1999). Distinct calcium dependent pathways of epidermal growth factor receptor transactivation and PYK2 tyrosine phosphorylation in PC12 cells. J. Biol. Chem. 274, 20989-20996.

39. Lowrie D. B., Tascon R. E., Bonato V. L., Lima V. M, Faccioli L. H., Stavropoulos E., Colston M. J., Hewinson R. G., Moelling K., Silva C. L. Therapy of tuberculosis in mice by DNA vaccination. Nature. (1999), Jul 15; 400:269-71.

40. Lowrie D. B., Silva C. L. Enhancement of immunocompetence in tuberculosis by DNA vaccination. Vaccine. (2000), Feb 25; 18:1712-1716.

41. Tanghe A., Lefevre P., Denis O., D'souza S., Braibant M., Lozes E., Singh M., Montgomery D., Content J., Huygen K. Immunogenicity and protective efficacy of tuberculosis DNA vaccines encoding putative phosphate transport receptors. J.lmmunol.(1999), Jan 15; 162:1113-9.

42. Baldwin S. L., D'souza C. D., Orme I. M., Liu M. A., Huygen K., Denis O., Tang A., Zhu L., Montgomery D., Ulmer J. B. Immunogenecity and protective efficacy of DNA vaccines encoding secreted and non secreted forms of Mycobacterium tuberculosis Ag 85A. Tuber.Lung Dis.(1999), 79; 251-9.

43. Kamath A. T., Feng C. G., Macdonald M., Briscoe H., Britton W. J. Differential protective efficacy of DNA vaccines expressing secreted proteins of Mycobacterium tuberculosis. Infect. Immun. (1999), 67; 1702-7.

44. Morris S., Kelley C., Howerd A., Li Z., Collins F. The immunogenecity of single and combination DNA vaccines against tuberculosis. Vaccine. (2000); Apr 1; 18, 2155-3.

1. A composition capable of inhibiting or preventing mycobacterial growth comprising an inhibitor of secretory tyrosine phosphatases from mycobacteria as an active agent and a pharmaceutically acceptable carrier.

2. The composition of claim 1, wherein said inhibitor is a selective inhibitor of mycobacterial tyrosine phosphatases.

3. The composition of claim 1 or 2, wherein said inhibitor is an antibody.

4. The composition of any one of claims 1-3, wherein said secretory tyrosine phosphatase is encoded by
(a) a nucleic acid comprising the nucleotide sequence as shown in SEQ ID NO:1 or a nucleic acid complementary thereto,
(b) a nucleic acid corresponding to the sequence of (a) within the scope of degeneracy of the genetic code or
(c) a nucleic acid which hybridizes under stringent conditions with a sequence of (a) and/or (b).

5. The composition of claim 4, wherein said secretory tyrosine phosphatase comprises the amino acid sequence as shown in SEQ ID NO:2.

6. The composition of any one of claims 1-3, wherein said secretory tyrosine phosphatase is encoded by
(a) a nucleic acid comprising the nucleotide sequence as shown in SEQ ID NO:3 or a nucleic acid complementary thereto,
(b) a nucleic acid corresponding to the sequence of (a) within the scope of degeneracy of the genetic code or
(c) a nucleic acid which hybridizes under stringent conditions with a sequence of (a) and/or (b).

7. The composition of claim 5, wherein said secretory tyrosine phosphatase comprises the amino acid sequence as shown in SEQ ID NO:4.

8. An immunogenic composition comprising a secretory tyrosine phosphatase from mycobacteria or an immunogenic fragment thereof.

9. An immunogenic composition comprising a nucleic acid encoding a secretory tyrosine phosphatase from mycobacteria or an immunogenic fragment thereof.

10. The composition of claim 8 or 9, wherein said secretory tyrosine phosphatase is encoded by
(a) a nucleic acid comprising the nucleotide sequence as shown in SEQ ID NO:1 or a nucleic acid complementary thereto,
(b) a nucleic acid corresponding to the sequence of (a) within the scope of degeneracy of the genetic code or
(c) a nucleic acid which hybridizes under stringent conditions with a sequence of (a) and/or (b).

11. The composition of claim 10, wherein said secretory tyrosine phosphatase comprises the amino acid sequence as shown in SEQ ID NO:2.

12. The composition of claim 8 or 9, wherein said secretory tyrosine phosphatase is encoded by
(a) a nucleic acid comprising the nucleotide sequence as shown in SEQ ID NO:3 or a nucleic acid complementary thereto,
(b) a nucleic acid corresponding to the sequence of (a) within the scope of degeneracy of the genetic code or
(c) a nucleic acid which hybridizes under stringent conditions with a sequence of (a) and/or (b).

13. The composition of claim 12, wherein said secretory tyrosine phosphatase comprises the amino acid sequence as shown in SEQ ID NO:4.

14. The composition of any one of claims 1-13, which is a pharmaceutical composition.

15. The composition of claim 14, which is a vaccine.

16. Use of a composition of any one of claims 1-15 for the manufacture of an agent for the inhibition or prevention or mycobacterial growth.

17. The use of claim 16 for the treatment or prevention of mycobacterial diseases.

18. The use of claim 16 or 17 for the treatment or prevention of tuberculosis.

19. An antibody against secretory tyrosine phosphatases from mycobacteria.

20. A method for the inhibition or prevention of mycobacterial growth comprising administering a composition of any one of claims 1-15 in an effective amount.

21. The method of claim 20 for the treatment or prevention of mycobacterial diseases.

22. The method of claim 20 or 21 for the treatment or prevention of tuberculosis.

23. A method for the detection of mycobacterial growth comprising contacting a sample suspected to contain mycobacteria or secretory products thereof with a reagent specific for secretory tyrosine phosphatases from mycobacteria.

24. A method of determining, if a test substance is an inhibitor of mycobacterial growth, comprising determining the effect of the test substance on a secretory phosphatase from mycobacteria.
